(19) United States
(12) Patent Application Publication
(10) Pub. No.: US 2006/0014939 A1
(43) Pub. Date: Jan. 19, 2006

(54) Novel C6-substituted furanoid sugar amino acids and improved process for preparing the same

(76) Inventors: Tushar Kanti Chakraborty, Hyderabad (IN); Gangarajula Sudhakar, Hyderabad (IN)

Correspondence Address: REED SMITH LLP, 2500 ONE LIBERTY PLACE, 1650 MARKET STREET, PHILADELPHIA, PA, 19103, US

(21) Appl. No.: 10/892,542

(22) Filed: Jul. 15, 2004

(51) Int. Cl. C07D 307/14; A61K 31/7008

(52) U.S. Cl. 536/17.1; 549/484; 536/18.7

(57) ABSTRACT

The present invention relates to furanoid sugar amino acids and their salts which carrying an additional chiral center at C6-position with substituents and resembling the side-chains of natural amino acids. More particularly, 6-substituted-6-deoxy-6-amino-2,5-anhydroaldonic acids and their 3,4-dideoxy congeners, in enantiomerically pure forms, which constitute an important class of conformationally constrained peptide building blocks that can be used as dipeptide isosteres in peptidomimetic studies.

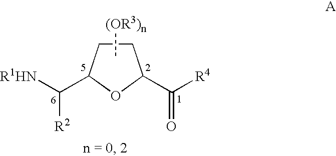

R¹=H, Boc, Cbz, Fmoc, acetyl or salts such as HCl, TFA

R²=CH₃—, (CH₃)₂CH—, (CH₃)₂CHCH₂—, CH₃CH₂CH(CH₃)—, alkyl groups, OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁵)CH₂—, (CO₂R⁵)CH₂CH₂—, Ph-, Ar-, PhCH₂—, ArCH₂—, Phenylalkyl-, arylalkyl-, (indolyl)CH₂—, (imidazolyl)CH₂—, and all other amino acid side-chains

R³=H, tert-butyl, alkyl, benzyl, arylCH₂, CO(alkyl), CO(arylalkyl), SO₃H, PO₃H₂, silyl,

R₄=—O-alkyl, —O-arylalkyl, -amine, -alkylamine, -arylalkylamine, and others

R₅=H, tert-butyl, alkyl, benzyl, arylCH₂,

R¹-R²=—(CH₂)ₙ— (n=2,3,4)

### FIELD OF INVENTION

The present invention relates to furanoid sugar amino acids and their salts which carrying an additional chiral center at C6-position with substituents and resembling the side-chains of natural amino acids. More particularly, the present invention relates to stereo selective preparation of C6-substituted furanoid sugar amino acids and their salts using chiral amino acids as starting materials.

### BACKGROUND OF THE INVENTION

There is large need of new molecular entities for discovering new drugs and materials. Organic chemists are looking for innovative approaches and trying to imitate nature and to assemble quickly large number of distinct and diverse molecular structures from ‘nature-like.’ The Researchers are using combinatorial approach with unnatural building blocks. The main objective in developing such libraries is to mimic the diversities displayed in structures and properties of natural products. The unnatural buildings blocks are used in these assemblies and carefully designed to manifest the structural diversities of the monomeric units. The building blocks are present in nature, like amino acids, carbohydrates and nucleosides to build its arsenal. Compounds made of such unnatural building blocks are also expected to be more stable toward proteolytic cleavage in physiological systems than their natural counterparts.

In recent years, sugar amino acids have emerged as one of such versatile templates which have been used extensively as conformationally constrained scaffolds in many peptidomimetic studies and as an important class of synthetic monomers, which are leading to many de novo oligometic libraries (Curr. Med Chem. 2002, 9, 421-435; Combinatorial Chem. High Throughput Screening 2002, 5, 373-387; Chem. Rev. 2002, 102, 491-514).

Sugar amino acids are basically hybrids of carbohydrates and amino acids. These designer building blocks are basically carbohydrate molecules bearing both amino and carboxyl functional groups on the regular 2,5-anhydro sugar frameworks. There are several advantages of sugar amino acids as building blocks.

(a) The rigid furan rings of these molecules make them ideal candidates as non-peptide scaffolds in peptidomimetics, where they can be easily incorporated by using their carboxyl and amino termini utilizing well-developed solid-phase or solution-phase peptide synthesis methods.

(b) At the same time, they allows efficient exploitation of the structural diversities of carbohydrate molecules to create combinatorial library of sugar amino acid based on molecular frameworks predisposed, to fold into architecturally beautiful ordered structures which may also have interesting properties.

(c) The protected/unprotected hydroxyl groups of sugar rings can also influence the hydrophobic/hydrophilic nature of such molecular assemblies.

Introduction of a chiral center in the amino terminus of these furan amino acids gives rise to an additional combinatorial site in these multifunctional building blocks that will not only help to induce desired secondary structure in peptides, but will also allow to mimic the side-chains of natural amino acids influencing the hydrophobicity/hydrophilicity of the resulting peptidomimetic molecules. Development of a robust synthetic strategy to construct these molecules in enantiomerically pure forms will allow their wide-ranging applications in peptidomimetic studies. Compounds with methyl substitution at the C6 position of 3,4-dideoxy furanoid sugar amino acids have been synthesized and used in peptidomimetic studies (Org. Biomol. Chem. 2003, 2983-2997; Angew. Chem. Int. Ed. 2000, 39, 900-902; Eur. J. Org. Chem. 1999, 2977-2990). However, the reported procedures suffers from poor diastereoselectivity leading to mixture of isomers and furthermore, the method was not amenable to prepare the 3,4-hydroxylated versions of these molecules. The present invention allows synthesis of all the stereoisomers of these molecules in enantiomerically pure forms. The process can be used not only for the synthesis of the 3,4-dideoxy variants of these molecules, but also capable to be extended to prepare their hydroxylated congeners.

The following abbreviation are used with the following meanings: Boc: tert-butoxycarbonyl; CSA: camphor sulphonic acid; DMSO: dimethyl sulfoxide; FmocOSu: 9-fluorenylmethyl N-succinimidyl carbonate; NMO: N-methylmorpholine N-oxide, PCC: pyridinium chlorochromate; TBAF: tetra-n-butylammonium fluoride; TBDPSCl: tert-butyldiphenylsilyl chloride; TFA: trifluoroacetic acid; TrisCl: 2,4,6-triisopropylbenzenesulfonyl chloride. The hidden line represents associated optional group i.e (OR³)n. Amino acids are denoted by L or D appearing before the symbol and separated from it by hyphen.

### OBJECTIVES OF THE INVENTION

The main objective of the present invention is to provide furanoid sugar amino acids (6-deoxy-6-amino-2,5-anhydroaldonic acids) which carry a chiral center at C6-position giving rise to an additional combinatorial site in said multifunctional building blocks.

Another objective of the present invention is to provide a process for preparing an important class of conformationally constrained chiral peptide building blocks which is used in peptidomimetic studies.

Yet another objective of the present invention is to provide an efficient synthetic strategies to prepare these molecules from inexpensive starting materials in pure chiral forms.

### SUMMARY OF THE INVENTION

The present invention relates to the stereoselective synthesis of C6-substituted furanoid sugar amino acids using chiral L- or D-amino acids and (R)- or (S)-glyceraldehyde acetonide as starting materials that give rise to two, C2 and C6, of the total five chiral centers of the molecule with the remaining three chiral centers, C3-C5, being built employing various stereoselective transformations.

These C6-substituted furanoid sugar amino acids constitute an important class of conformationally constrained chiral peptide building blocks that can be used as dipeptide isosteres in peptidomimetic studies and also an important class of combinatorial building blocks with five chiral centers that can give rise to 32 distinct stereoisomers each with four combinatorial sites.

### DETAILED DESCRIPTION OF THE INVENTION

Accordingly the present invention relates to chiral furanoid sugar amino acids of peptide compound carrying an additional chiral center at C6-position with substituents, resembling the side-chains of natural amino acids and having a general structure as shown in FIG. 1 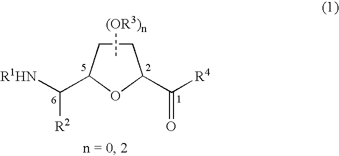
Wherein;
R¹=H, tert-butoxycarbonyl (Boc), benzyloxycarbonyl (Cbz), 9-fluroenylmethyl (Fmoc), acetyl or their salts, their such as hydrochloric acid (HCl), tri fluro accetic acid (TFA)
R²=CH₃—, (CH₃)₂CH—, (CH₃)₂CHCH₂—, CH₃CH₃CH(CH₃)—, alkyl groups, (OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁵)CH₂—, (CO₂R⁵)CH₂CH₂—, Ph-, Ar-, PhCH₂—, ArCH₂—, Phenylalkyl-, arylalkyl-, (indolyl)CH₂—, (imidazolyl)CH₂—, and all other amino acid side-chains
R³=H, tert-butyl, alkyl, benzyl, arylCH₂, CO(alkyl), CO(arylalkyl), SO₃H, PO₃H₂, silyl,
R⁴=—O-alkyl, —O-arylalkyl, -amine, -alkylamine, -arylalkylamine, and others
R⁵=H, tert-butyl, alkyl, benzyl, arylCH₂,
R¹-R²=—(CH₂)ₙ— (n=2, 3, 4)

In an embodiment of the present invention wherein if the stereochemistry of C₆ is S form and the substitutions are R¹=Boc, R²=Me and R⁴=Me compound having structural formula 2

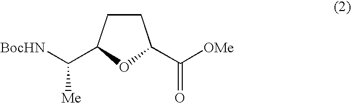

In another embodiment of the present invention wherein if the stereo chemistry of C₆ is S form and the substitutions are R¹=Boc, R²=Me and R⁴=H compound having structural formula 3

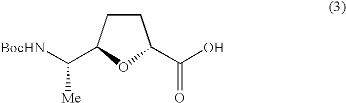

In yet another embodiment of the present invention wherein if the stereochemistry of C₆ is S form and the substitutions are and R¹=CF₃COOH.H, R²=Me and R⁴=Me compound having structural formula 4

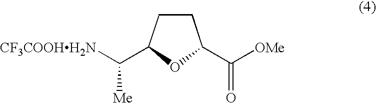

In one another embodiment of the present invention wherein if the stereochemistry of C₆ is S form and the substitutions are and R¹=CF₃COOH.H, R²=Me and R⁴=H compound having structural formula 5

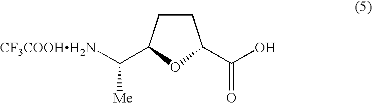

In yet another embodiment of the present invention wherein if the stereochemistry of C₆ is R form and the substitutions are and R¹=Boc, R²=Me and R⁴=Me compound having structural formula 6

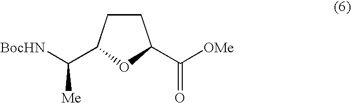

In still another embodiment of the present invention wherein if the stereochemistry of C₆ is R form and the substitutions are R¹=Boc, R²=Me and R⁴=H compound having structural formula 7

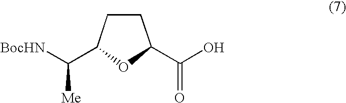

In a further embodiment of the present invention wherein if the stereochemistry of C₆ is R form and the substitutions are R¹=CF₃COOH.H, R²=Me and R⁴=Me compound having structural formula 8

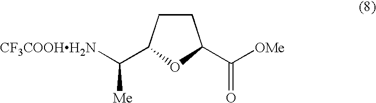

In a further another embodiment of the present invention wherein if the stereochemistry of C₆ is R form and the substitutions are R¹=CF₃COOH.H, R²=Me and R⁴=H compound having structural formula 9

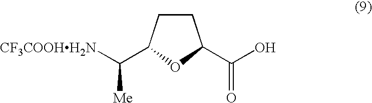

In yet another embodiment of the present invention wherein if the stereochemistry of C₆ is S form and the substitutions are R¹=Boc, R²=Me and R⁴=Me compound having structural formula 10

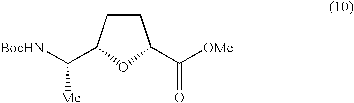

In yet another embodiment of the present invention wherein if the stereochemistry of C₆ is S form and the substitutions are R¹=Boc, R²=Me and R⁴=H compound having structural formula 11

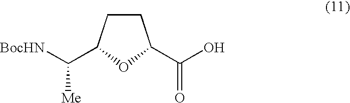

In still another embodiment of the present invention wherein if the stereochemistry of C₆ is S form and the substitutions are R¹=CF₃COOH.H R²=Me and R⁴=Me compound having structural formula 12

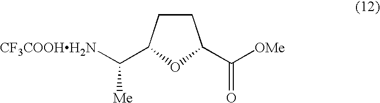

In one more embodiment of the present invention wherein if the stereochemistry of C₆ is S form and the substitutions are R¹=CF₃COOH.H, R²=Me and R⁴=H compound having structural formula 13

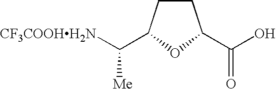

In yet another embodiment of the present invention wherein if the stereochemistry of C₆ is R form and the substitutions are R¹=Boc, R²=Me and R⁴=Me compound having structural formula 14

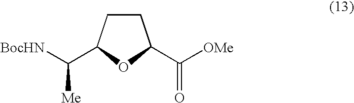

In yet another embodiment of the present invention wherein if the stereochemistry of C₆ is S form and the substitutions are R¹=Boc, R²=Me and R⁴=H compound having structural formula 15

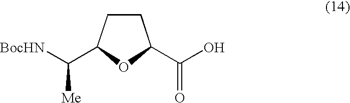

In a further embodiment of the present invention wherein if the stereochemistry of C₆ is R form and the substitutions are R¹=CF₃COOH.H, R²=Me and R⁴=Me compound having structural formula

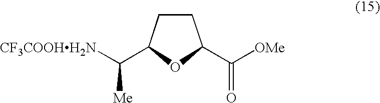

In a further more embodiment of the present invention wherein if the stereochemistry of C₆ is R form and the substitutions are R¹=CF₃COOH.H, R²=Me and R⁴=H compound having structural formula 16

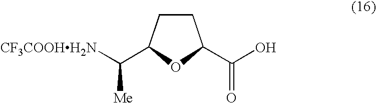

In another embodiment of the present invention wherein if the stereochemistry of C₆ is S form and the substitutions are R¹=Boc, R²=Me and R⁴=Me compound having structural formula 17

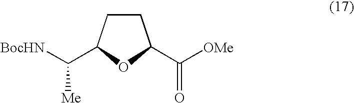

In yet another embodiment of the present invention wherein if the stereochemistry of C₆ is S form and the substitutions are R¹=Boc, R²=Me and R⁴=H compound having structural formula 18

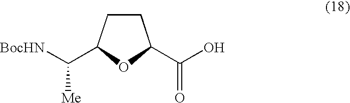

In yet another embodiment of the present invention wherein if the stereochemistry of C₆ is R form and the substitutions are R¹=CF₃COOH.H, R²=Me and R₄=Me compound having structural formula 19

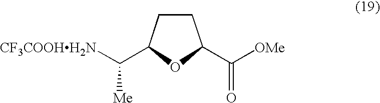

In yet another embodiment of the present invention wherein if the stereochemistry of C₆ is S form and the substitutions are R¹=CF₃COOH.H, R²=Me and R⁴=H compound having structural formula 20

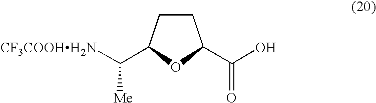

In yet another embodiment of the present invention wherein if the stereochemistry of C₆ is R form and the substitutions are R¹=Boc, R²=Me and R⁴=Me compound having structural formula 21

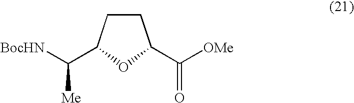

In yet another embodiment of the present invention wherein if the stereochemistry of C₆ is R form and the substitutions are R¹=Boc, R²=Me and R⁴=H compound having structural formula 22

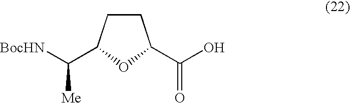

In yet another embodiment of the present invention wherein if the stereochemistry of C₆ is R form and the substitutions are R¹=CF₃COOH.H, R²=Me and R⁴=Me compound having structural formula 23

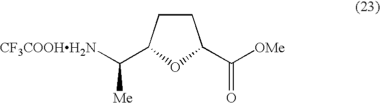

In still another embodiment of the present invention wherein if the stereochemistry of C₆ is R form and the substitutions are R¹=CF₃COOH.H, R²=Me and R⁴=H compound having structural formula 24

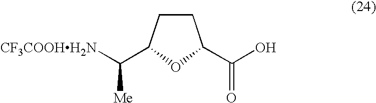

In still another embodiment of the present invention wherein if the stereochemistry of C₆ is S form and the substitutions are R¹=Boc, R²=Me and R⁴=Me compound having structural formula 25

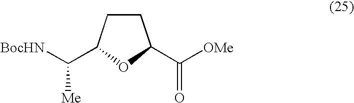

n one more embodiment of the present invention wherein if the stereochemistry of C₆ is S form and the substitutions are R¹=Boc, R² =Me and R⁴=H compound having structural formula 26

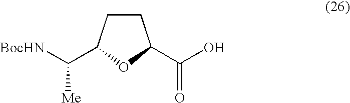

In one another embodiment of the present invention wherein if the stereochemistry of C₆ is S form and the substitutions are R¹=CF₃COOH.H, R²=Me and R⁴=Me compound having structural formula 27

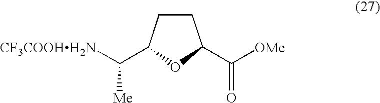

In yet another embodiment of the present invention wherein if the stereochemistry of C₆ is S form and the substitutions are R¹=CF₃COOH.H, R²=Me and R⁴=H compound having structural formula 28

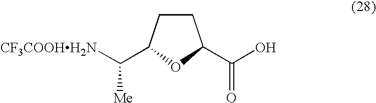

In a further embodiment of the present invention wherein if the stereochemistry of C₆ is S form and the substitutions are R¹=CF₃COOH.H, R²=Me and R⁴=H compound having structural formula 29

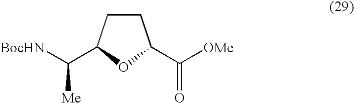

In a further more embodiment of the present invention wherein if the stereochemistry of C₆ is R form and the substitutions are R¹=Boc, R²=Me and R⁴=H compound having structural formula 30

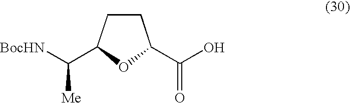

In yet another embodiment of the present invention wherein if the stereochemistry of C₆ is R form and the substitutions are R¹=CF₃COOH.H, R²=Me and R⁴=Me compound having structural formula 31

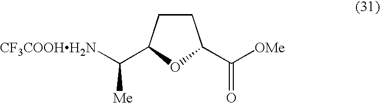

In yet another embodiment of the present invention wherein if the stereochemistry of C₆ is R form and the substitutions are R¹=CF₃COOH.H, R²=Me and R⁴=H compound having structural formula 32

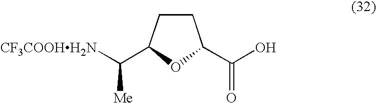

In still another embodiment of the present invention wherein if the stereochemistry of C₆ is S form and the substitutions are R¹=Boc, R²=Me and R⁴=Me compound having structural formula 33

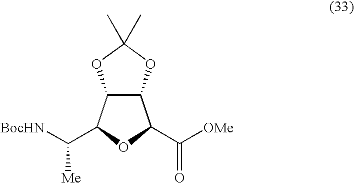

In one more embodiment of the present invention wherein if the stereochemistry of C₆ is S form and the substitutions are R¹=Boc, R²=Me and R⁴=H compound having structural formula 34

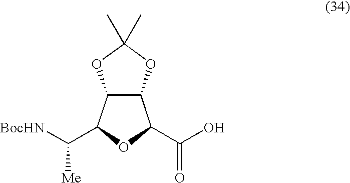

In one another embodiment of the present invention wherein if the stereochemistry of C₆ is S form and the substitutions are R¹=CF₃COOH.H, R²=Me and R⁴=Me compound having structural formula 35

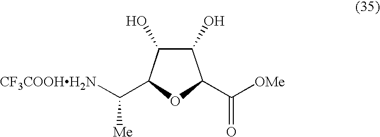

In yet another embodiment of the present invention wherein if the stereochemistry of C₆ is S form and the substitutions are R¹=CF₃COOH.H, R²=Me and R⁴=H compound having structural formula 36

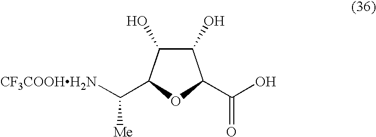

In yet another embodiment of the present invention wherein if the stereo chemistry of C₆ is S form and the substitutions are R¹=Boc, R²=Me and R⁴=H having following characteristics R_{f}=0.45 (silica, 1:3 ethyl acetateahexane); ¹H NMR (200 MHz, CDCl₃): δ 4.67 (d, J=6.6, 1H, NH) 4.54 (dd, J=5.2, 8.1 Hz, 1 H, C2H), 4.15 (m, 1H, C5H), 3.74 (s, 3H, CO₂Me), 3.71 (m, 1H, C₆H), 2.28 (m, 1H), 2.03 (m, 1H), 1.72 (m, 2H) 1.44 (s, 9H, t-butyl) 1.14 (d, J=6.6 Hz, 3H, C6CH₃).

In still another embodiment of the present invention relates to a Process for preparing C₆-substituted chiral furanoid sugar amino acids of peptide compound, carrying natural amino acid side-chains and having a general structure 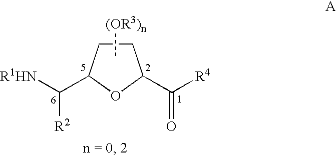
Wherein;
R¹=H, tert-butoxycarbonyl (Boc), benzyloxycarbonyl (Cbz), 9-fluroenylmethyl (Fmoc), acetyl or their salts made of acid such as hydrochloric acid (HCl), tri fluro accetic acid (TFA),
R²=CH₃—, (CH₃)₂CH—, (CH₃)₂CHCH₂—, CH₃CH₂CH(CH₃)—, alkyl groups, (OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁵)CH₂—, (CO₂R⁵)CH₂CH₂—, Ph-, Ar-, PhCH₂—, ArCH₂—, Phenylalkyl-, arylalkyl-, (indolyl)CH₂—, (imidazolyl)CH₂—, and all other amino acid side-chains
R³=H, tert-butyl, alkyl, benzyl, arylCH₂, CO(alkyl), CO(arylalkyl), SO₃H, PO₃H₂, silyl,
R⁴=—O-alkyl, —O-arylalkyl, -amine, -alkylamine, -arylalkylamine, and others
R⁵=H, tert-butyl, alkyl, benzyl, arylCH₂,
R¹-R²—(CH₂)ₙ— (n=2, 3, 4)
said process comprising the steps of:
a) addition of L- or D N,N-dibenzylamino aldehydes, prepared in-situ by reacting 3,4-O-isopropylidene-1,1-dibromobut-1-en-3,4diol with n-BuLi, to the N,N-dibenzyl amino aldehyde to give the propargylic alcohol adducts, 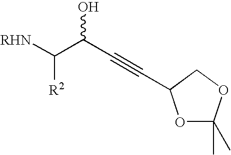
b) hydrogeneation of the adduct obtained in step (a) in presence of Pd(OH)₂—C catalyst and in the presence of acid to deprotect (i) N-terminus, (ii) acetonide and (iii) reduce the triple bond, all in one pot and to get an intermediate of N-Boc-protected,
(c) intermediate of step (b) dissolved in MeOH, neutralize with Et₃N and Bo.C₂O and stirring the same for a period of 3 hours to obtain triol as a intermediate compound, 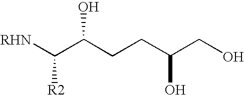
(d) mixing 2,4,6 tri iso propyl benzene chloride with triol of step (c) to obtain sulfonated intermediate,
(e) mixing sulfonted intermediate of step (d) with water in presence of MeOH and K₂CO₃, extract the same in EtOAc, 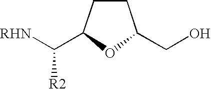
(f) dissolving intermediate compound of step (e) in presence of CH₂Cl & DMSO and Et₃N/SO₃-Py complex, obtaining organic layer & aqueous layer, washing with water to obtain a brine solution.
(g) brine solution of step (f) dissolved in 2-methyl 2-Butene and t-BuOH & in presence of NaClO₂, NaPO₄, acidifying with HCl to obtain desired product.

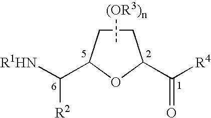

In an embodiment of the present invention wherein in step (g), if C₆ is S form and R¹=Boc, R²=Me and R⁴=H compound having structural formula

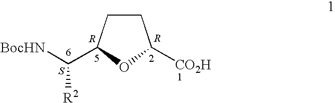

In another embodiment of the present invention wherein if C₆ is S form and R¹=Boc, R²=Me and R⁴=H compound 3 having structural formula

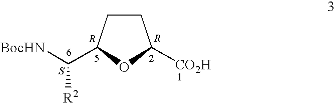

In one embodiment of the present invention wherein if C₆ is R form and R¹=Boc, R²=Me and R⁴=H compound 5 having structural formula

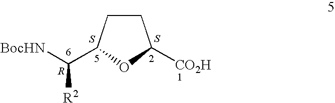

In one more embodiment of the present invention wherein if C₆ is S form and R¹=Boc, R²=Me and R⁴=H compound 7 having structural formula

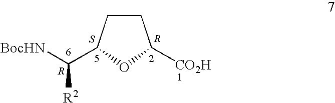

In one another embodiment of the present invention wherein said process comprising the steps of wherein if C₆ is S form and R¹=Boc, R²=Me and R⁴=H compound having structural formula

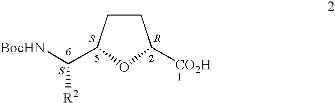

In yet another embodiment of the present invention wherein intermediate compound obtained in step (a) is oxidized to a keto intermediate using SO₃-Py and reducing in presence of k-selectride to get the hydroxyl bearing centre inverted intermediate having structure (12), and following the steps (b) to (i) to obtain a compound 2, 4, 6 and 8.

In one embodiment of the present invention wherein the intermediate structure 12 having general formula

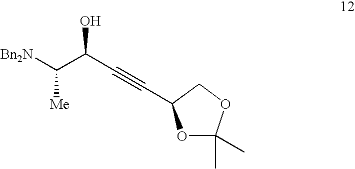

In yet another embodiment of the present invention wherein if C₆ is S form and R¹=Boc, R²=Me and R⁴=H compound 2 having structural formula

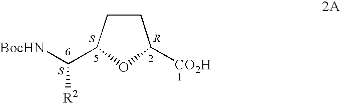

In yet another embodiment of the present invention wherein if C₆ is S form and R¹=Boc, R²=Me and R⁴=H compound 4 having structural formula

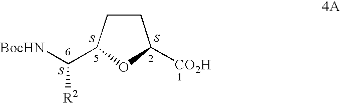

In yet another embodiment of the present invention wherein if C₆ is S form and R¹=Boc, R²=Me and R⁴=H compound 6 having structural formula

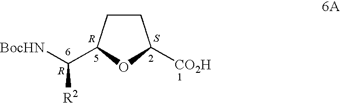

In yet another embodiment of the present invention wherein if C₆ is R form and R¹=Boc, R²=Me and R⁴=H compound 8 having structural formula

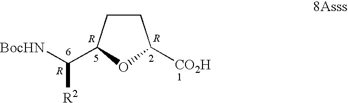

In yet another embodiment of the present invention wherein for chiral furamn amino acid compounds 5, 9, 13, 17, 21, 25, 29, 33 and 37 treatment with FmocOSu in dioxane-water (1:1) is carried out to give N-Fmoc protected C₆-substituted furanoid sugar amino acid. Accordingly, the present invention relates to the development of an efficient method for the stereoselective construction of C₆-substituted furanoid sugar amino acids, an important class of combinatorial building blocks, as shown in general structure A in Formula 1, using commercially available chiral N-protected amino aldehydes as starting materials that could also be prepared from the corresponding L- or D-amino acids. 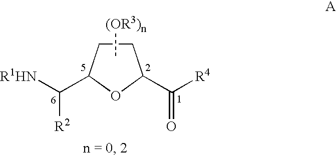
R¹=H, Boc, Cbz, Fmoc, acetyl or their salts made of such as HCl, TFA and others R²=CH₃—, (CH₃)CH—, (CH₃)₂CHCH₂—, CH₃CH₂CH(CH₃)—, alkyl groups, (OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁵)CH₂—, (CO₂R⁵)CH₂CH₂—, Ph-, Ar-, PhCH₂—, ArCH₂—, Phenylalkyl-, arylalkyl-, (indolyl)CH₂—, (imidazolyl)CH₂—, and all other amino acid side-chains
R³=H, tert-butyl, alkyl, benzyl, arylCH₂, CO(alkyl), CO(arylalkyl), SO₃H, PO₃H₂, silyl,
R⁴=—O-alkyl, —O-arylalkyl, -amine, -alkylamine, -arylalkylamine, and others
R⁵=H, tert-butyl, alkyl, benzyl, arylCH₇,
R¹-R²=—(CH₂)ₙ— (n=2, 3, 4)

### FORMULA 1

Synthesis of C₆-Substituted Furanoid Sugar Amino Acids

The synthetic protocols developed in the present invention can suitably be employed to synthesize any of the 32 stereoisomers, having a general structure A as shown in Formula 1, in optically pure form, following an efficient route, in which chiral L- or D-amino acids and (R)- or (S)-glyceraldehyde acetonide are used as starting materials.

In the present invention, first of all, the 8 possible stereoisomers of C₆-substituted 3,4-dideoxyfuranoid sugar amino acids 1-8 (n=0, structure A in Formula 1) as depicted in Formula 2 were synthesized starting from chiral amino acids. Among these isomers, 1 and 5, 2A and 6A, 3 and 7, 4A and 8A are enantiomeric pairs. The first four of these compounds 1-4 were prepared from L-amino acids, the remaining ones 5-8 were made using D-amino acids as starting materials.

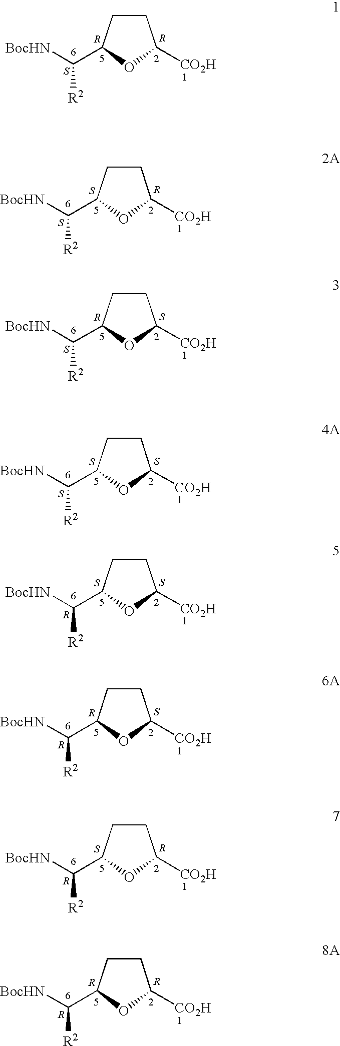

The outlines of the synthetic schemes for the first two compounds 1-2 are shown in Scheme 1. The starting materials in this scheme were L-amino acid derived N,N-dibenzylamino aldehyde 9 and 3,4-O-isopropylidene-1,1-dibromobut-1-en-3,4-diol 10 that was prepared from (R)-glyceraldehyde acetonide, which could be made easily in large quantities by oxidative cleavage of 1,2:5,6-di-O-isopropylidene-D-mannitol using NaIO₄ (Gung, B. W. etal J. Org. Chem. 2003, 68, 5956-5960; Schmid, C. R. etal J. Org. Chem. 1991, 56, 4056-4058). Treatment of 9 with the Li-acetylide prepared in-situ by reacting 3,4-O-isopropylidene-1,1-dibromobut-1-en-3,4-diol 10 (Gung, B. W. etal J. Org. Chem. 2003, 68, 5956-5960) with n-BuLi, gave the adduct 11 stereoselectively. The stereochemistry of the newly generated hydroxyl group was reversed by subjecting 11 to an oxidation-reduction sequence to give the isomeric product 12. Compounds 11 and 12 were finally transformed into the furanoid 3,4-dideoxy sugar amino acids (2R,5R,6S)-1 and (2R,5S)-2, respectively.

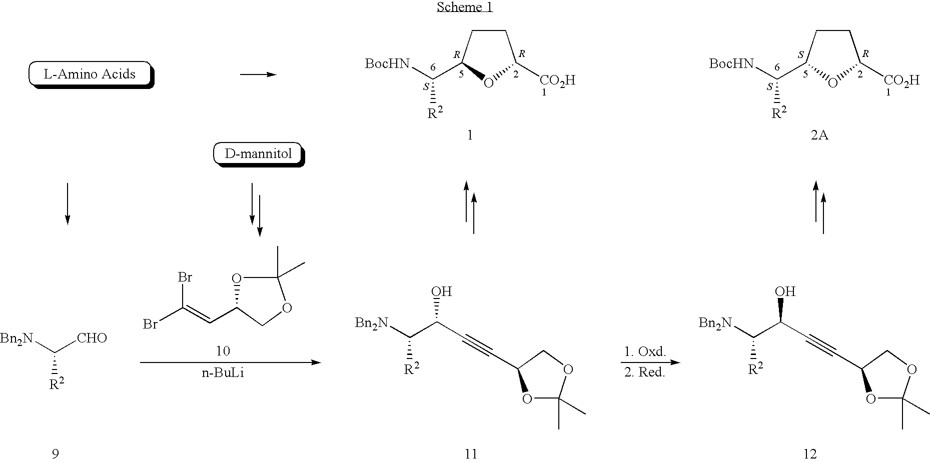

For the synthesis of compounds 3-4, as shown in Scheme 2, the same N,N-dibenzylamino aldehyde 9 used in Scheme 1 was reacted with the Li-acetylide prepared from isomeric 3,4-O-isopropylidene-1,1-dibromobut-1-en-3,4-diol 13. Compound 13 is an enantiomer of 10 that was prepared by known methods from L-ascorbic acid (Hubschwerlen, C. Synthesis 1986, 962; Takano, S. etal Heterocycles 1982, 19, 32). The adducts 14 and 15 were then converted into (2S,5R,6S)-3 and (2S,5S,6S)-4, respectively, following the same methods used in Scheme 1.

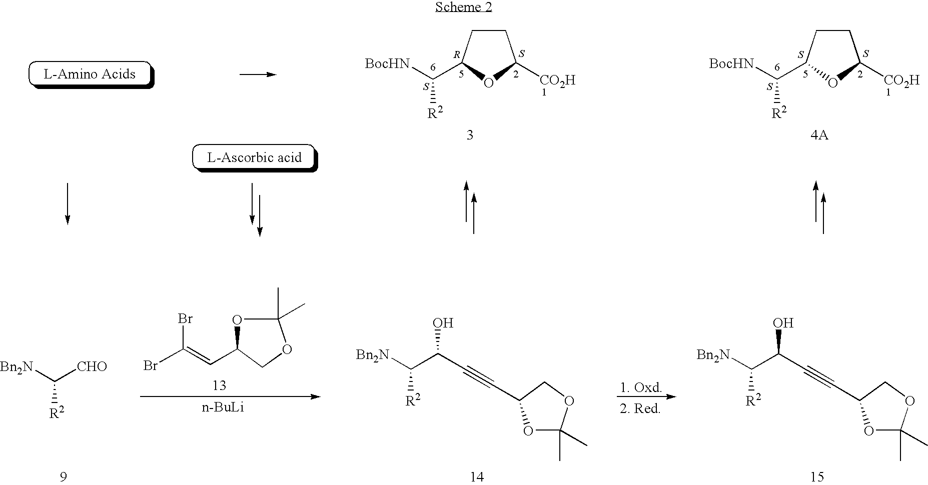

Similarly, starting from D-amino acids and following the same methods as outlined in Schemes 1 and 2, the other four isomers of this C₆-substituted 3,4-dideoxy furanoid sugar amino acids 5-8 were prepared as shown in Scheme 3.

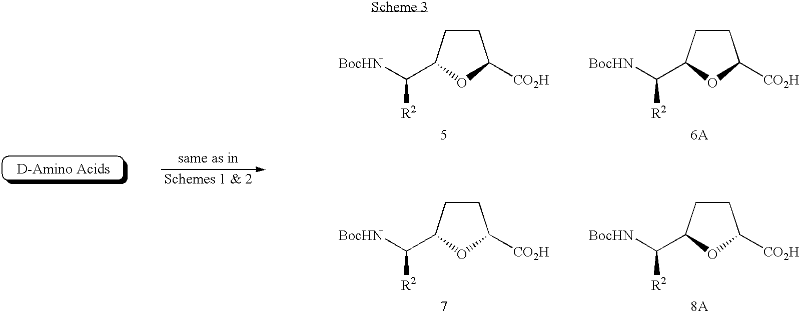

Thus, the present method as depicted in Schemes 1-3 gave all the eight possible isomers of C₆-substituted 3,4-dideoxyfuranoid sugar amino acids 1-8 in pure enantiomeric forms by just altering the chiralities of the starting amino aldehydes and glyceraldehyde acetonides, but essentially following a common strategy for all of them.

The approach described in Schemes 1-3 was further extended to synthesize the 3,4-dihydroxylated versions of these molecules (n=2, structure A in Formula 1), i.e., the regular C₆-substituted furanoid sugar amino acids, as shown in Scheme 4.

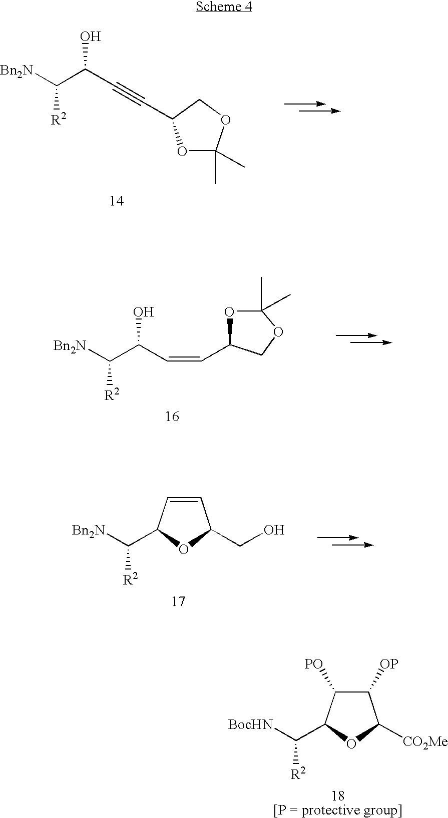

The adduct 14 from Scheme 2 was selectively reduced into a cis-allylic alcohol by hydrogenation using Landlar's catalyst as shown on Scheme 4. The resulting Z-olefinic compound 16 was transformed into the cyclised intermediate 17 following the same methods described in Schemes 1 and 2. Compound 17 was subjected to cis-hydroxylation to introduce the hydroxyl groups at C3 and C4 positions and eventually transformed into the final product, C₆-substituted 6-amino-deoxy-2,5-anhydro-D-aldonic acid 18 following the method described in Scheme 1. Similar strategy as outlined in Scheme 4 was followed to prepare the other isomers of the 3,4-dihydroxylated versions of these molecules.

Synthesis of 1 (R²=Me)

The various steps involved in Scheme 1 are shown in details in Scheme 5 with one of the substrates having R₂=Me. Synthesis of compound 1 (R²=Me) involved seven steps as can be seen in Scheme 5. Treatment of 9 (R²=Me) (Reetz, M. T. etal Org. Synth. 1998, 76, 110; Reetz, M. T. Chem. Rev. 1999, 99, 1121-1162) with the Li-acetylide prepared in-situ by reacting 3,4-O-isopropylidene-1,1-dibromobut-1-en-3,4-diol 10 (Gung, B. W. etal J. Org. Chem. 2003, 68, 5956-5960) with n-BuLi, gave the adduct 11 (R²=Me) stereoselectively. Hydrogenation of 11 (R²=Me) using 20% Pd(OH)₂—C as catalyst in MeOH containing HCl reduced the triple bond, deprotected both NBn₂ and acetonide giving an amino triol intermediate, which was protected at the N-terminal using Boc₂O to furnish the triol 19.

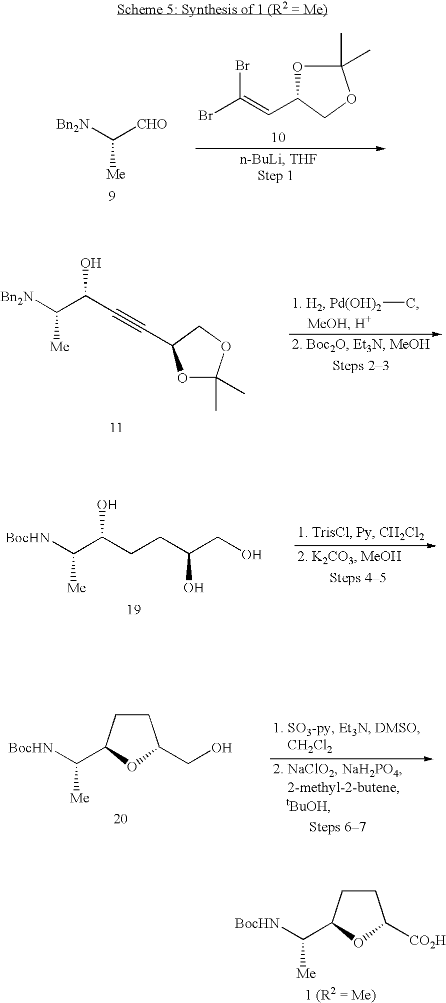

Selective sulfonylation of the primary hydroxyl group using 2,4,6-triisopropylbenzenesulfonyl chloride (TrisCl) gave a sulfonate intermediate (Maezaki, N. etal Org. Lett. 2002, 4, 2977-2980) that was treated with anhydrous K₂CO₃ to carry out a facile intramolecular ring closure reaction via an epoxide intermediate to get the tetrahydrofuran framework of 20. Finally, a two-step oxidation process converted the primary hydroxyl group of 20 into the desired product 1 (R²=Me).

Step 1

To a solution of the dibromo compound 10 (20 g) (Gung, B. W. etal J. Org. Chem. 2003, 68, 5956-5960) in dry THF at −78° C., nBuLi (83 mL, 1.6 M in hexane) was added drop wise and the solution was stirred at −78° C. for 30 min and at room temperature for 30 min. Again the reaction mixture was cooled to −78° C. and to this, a solution of the aldehyde 9 (14.8 g) (Reetz, M. T. etal Org. Synth. 1998, 76, 110; Reetz, M. T. Chem. Rev. 1999, 99, 1121-1162) in dry THF was added drop wise and stirred for 30 min at −78° C. The reaction mixture was quenched with saturated NH₄Cl solution, the organic layer was separated and the aqueous layer was extracted with EtOAc. The combined organic extracts were washed with water, brine, dried (Na₂SO₄) and concentrated in vacuo. Purification by column chromatography afforded compound 11 (R²=Me) in 81% yield (18 g). Data for 11: R_{f}=0.45 (silica, 1:1.5 ethyl acetate/hexane): ¹H NMR (500 MHz, CDCl₃): δ 7.33-7.22 (m, 10H, ArH), 4.7 (n, 1H, C2H), 4.2I (m, 1H, C2H) 4.1 (ABq, 2 H, NCH₂Ph), 4.08 (dd, J=6.5, 7.5 Hz, 1H, C1H), 3.91 (br, 1H, OH), 3.86 (dd, J=6.5. 8.1 Hz, 1H, C1H′), 3.38 (ABq, 2H, NCH₂Ph), 3.03 (m, 1H, C6H), 1.41 (a, 3H, acetonide Me), 1.33 (s, 3H, acetonide Me), 1.2 (d, J=6.7 Hz, 3H, C6CH₃).

Steps 2 and 3

Compound 11 (13 g) in MeOH was hydrogenated on 20% Pd(OH)₂/C (130 mg) in acidic medium (conc.HCl, 1% v/v) with stirring at room temperature for 12 h. The Pd(OH)₂/C was filtered off and the filtrate was concentrated under reduced pressure. The residue was dissolved in MeOH and the solution was cooled to 0° C. Then neutralized with Et₃N (9.56 mL) and Boc₂O (7.88 g) was added and stirred for 3 h at this temperature. The reaction mixture was quenched with saturated NH₄Cl solution. The MeOH was concentrated under reduced pressure. Then the aqueous layer was extracted with EtOAc and the EtOAc layer was washed with water, brine, dried (Na₂SO₄) and concentrated in vacuo. Purification by column chromatography (silica, 10% MeOH in CHCl₃ eluant) afforded compound 19 in 85% yield (7.8 g). Data for 19: R_{f}=0.45 (silica, ethyl acetate); ¹H NMR (300 MHz, CDCl₃); δ 5.08 (d, J=6.7 Hz, 1H, NH), 3.72 (m, 1H, C2H), 3.6 (m, 2H, C1H), 3.45 (m, 1H, C5H), 3.11 (m, 1H, C6H), 1.68-1.45 (m, 4H), 1.43 (s, 9H, t-butyl), 1.09 (d, J=6.4 Hz, 3H, C6CH₃).

Steps 4 and 5

2,4,6-Triisopropylbenzene chloride (14.28 g) was added to a solution of the triol 19 (3.1 g) in pyridine:CH₂Cl₂ (1:2) with stirring at 0° C. After 36 h at room temperature, water was added to the reaction mixture and the reaction mixture was extracted with EtOAc. The combined organic layers were washed with water, brine, dried (Na₂SO₄) and concentrated in vacuo. The residue was chromatographed on silica gel with hexane-EtOAc (6:4) as eluant to give a sulfonate intermediate in 74% yield (4.6 g)

To a solution of the sulfonate (4 g) in MeOH, anhydrous K₂CO₃ (1.56 g) was added with stirring at 0° C. The stirring was continued at room temperature for 8 h. Water was added to the reaction mixture and the mixture was extracted with EtOAc. The combined organic layers were washed with water, brine, dried (Na₂SO₄) and concentrated in vacuo. Purification by column chromatography afforded compound 20 in 97% yield (1.98 g). Data for 20: R_{f}=0.45 (silica, 1.5:1 ethyl acetate/hexane); ¹H NMR (500 MHz, CDCl₃): δ 4.62 (br, 1H, NH), 4.1(m, 1H, C2H), 3.94 (m, 1H, C5H), 3.7 (m, 1H, C6H), 3.65 (dd, J=2.9, 11.7 Hz, 1H, C1H), 3.48 (dd, J=5.8, 11.7 Hz, 1H, C1H), 2.04-1.94 (m, 2H), 1.78-1.66 (m, 2H), 1.44 (s, 9H, t-butyl), 1.13 (d, J=6.4 Hz, 3H, C6CH₃).

Steps 6 and 7

Compound 20 (1 g) was dissolved in dry CH₂Cl₂ and DMSO (1:1.25, 14.4 mL) and cooled to 0° C. Then Et₃N (2.84 mL) followed by SO₃-Py complex (3.25 g) were added and stirred 30 min at 0° C. The reaction was quenched with saturated NH₄Cl solution, the organic layer was separated and the aqueous layer was extracted with EtOAc. The combined organic layers were washed with water, saturated CuSO₄ solution, brine, dried (Na₂SO₄) and concentrated in vacuo.

The residue was dissolved in 2-methyl-2-butene and ¹BuOH (1:2, 12 mL). To this, a mixture of NaClO₂ (553 mg) and NaHPO₄ (955 mg) dissolved in distilled water were added at room temperature. Stirring continued for 1 h and then again a mixture of NaClO₂ (369 mg) and NaHPO₄ (636 mg) dissolved in distilled water were added and stirred for another 1 h. The solvents were evaporated in vacuo. The residue was dissolved in EtOAc, acidified with 1N HCl (pH=2), washed with water, brine, dried (Na₂SO₄) and concentrated. Purification by column chromatography afforded compound 1 (R²=Me) in 84% yield (0.9 g). Data for 1 (R=Me) methyl ester: R_{f}=0.45 (silica, 1:3 ethyl acetate/hexane); ¹H NMR (200 MHz, CDCl₃): δ 4.67 (d, J=6.6, 1H, NH). 4.54 (dd, J=5.2, 8.1 Hz, 1H, C2H), 4.15 (m, 1H, C5H), 3.74 (s, 3H, CO₂Me), 3.71 (m, 1H, C6H), 2.28 (m, 1H), 2.03 (m, 1H), 1.72 (m, 2H) 1.44 (s, 9H, t-butyl) 1.14 (d, J=6.6 Hz, 3H, C6CH₃).

Synthesis of 2A (R²=Me)

Compound 2A (R²=Me) was synthesized from 11 (R²=Me) in eight steps as shown in Scheme 6. The hydroxyl group of 11 (R²=Me) was oxidized to a keto intermediate using SO₃-Py and the resulting keto group was then reduced using K-selectride to get the hydroxyl-beating, center inverted. The resulting product 12 (R²=Me) was finally converted into the target molecule 2A (R²=Me) following the same six steps that were used in Scheme 5 to transform 11 into 1. 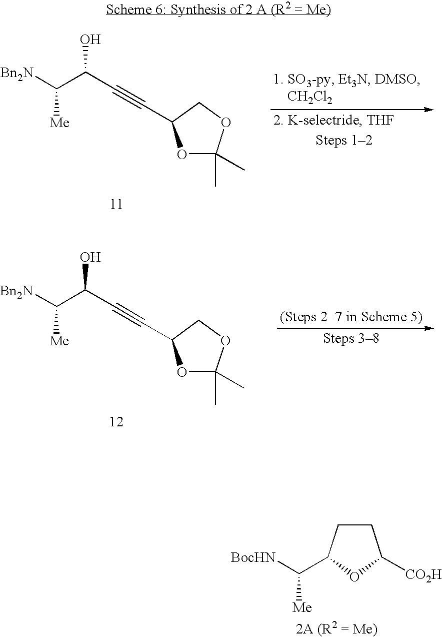
Steps 1 and 2

To a solution of compound 11 (3.2 g) in DMSO:CH₂Cl₂ (1.25:1, 28.8 mL) at 0° C., triethylamine (5.88 mL) was added followed by the addition of SO₃-Py complex (6.72 g) and stirred for 30 min. The reaction was then quenched with saturated NH₄Cl solution, the organic layer was separated and the aqueous layer was extracted with EtOAc. The combined organic extracts were washed with water, saturated CuSO₄ solution, brine, dried (Na₂SO₄) and concentrated in vacuo.

The residue was dissolved in dry THE and the solution was cooled to −78° C. A solution of K-selectride (8.5 ml, 1 M solution in THF) was added drop wise and the solution was stirred 30 min at −78° C. At 0° C., methanol (8.5 mL), 1N NaOH (8.5 mL) and 30% H₂O₂ (8.5 mL) were added sequentially and stirring continued for 2 h. The reaction was then quenched with saturated NH₄Cl solution, the organic layer was separated and the aqueous layer was extracted with EtOAc. The combined organic extracts were washed with water, brine, dried (Na₂SO₄) and concentrated in vacuo. Purification by column chromatography afforded compound 12 in 75% yield (2.4 g). Data for 12: R_{f}=0.45 (silica, 1:3 ethyl acetate/hexane); ¹H NMR (500 MHz, CDCl₃): δ 7.34-7.22 (m, 10H, ArH), 4.71 (dt, J=1.2, 6.6 Hz, 1H, C2H), 4.21 (dd, J=1.2, 9.6 Hz, 1H, C5H), 4.1 (dd, J=6.6, 7.8 Hz, 1 H, C1H), 3.85 (dd, J=6.6, 8.4 Hz, 1H, C1H′), 3.78 (ABq, 2H, NCH₂Ph), 3.36 (ABq, 2 H, NCH₂Ph), 288 (dq, J=6.6, 9.6 Hz, 1H, C₆H), 1.59 (br, 1H, OH), 1.44 (s, 3H, acetonide Me), 1.35 (s, 3H, acetonide Me), 1.17 (d, J=6.6 Hz, 3H, C6CH₃).

Steps 3-8

Compound 12 (R²=Me) was transformed into 2A (R²=Me) in 6 steps following the same procedures as described in the synthesis of 1 from 11 (Scheme 5).

Synthesis of 3 (R²=Me)

The seven steps involved in the synthesis of 3 (R²=Me) are shown in details in Scheme 7. Treatment of 9 (R²=Me) with the Li-acetylide prepared in-situ by reacting 3,4-O-isopropylidene-1,1-dibromobut-1-en-3,4-diol 13 prepared from L-ascorbic acid (Hubschwerlen, C. Synthesis 1986, 962; Takano, S. etal Heterocycles 1982, 19, 32) with n-BuLi gave the adduct 14 (R²=Me) stereoselectively. The resulting product 14 R²=Me) was finally converted into the target molecule 3 (R²=Me) following the same six steps that were used in Scheme 5 to transform 11 into 1.

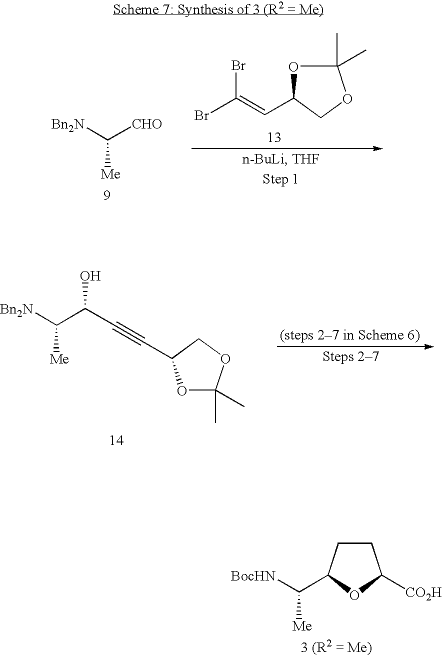

The 3,4-O-isopropylidene-1,1-dibromobut-1-en-3,4-diol 13 was prepared from the corresponding (S)-glyceraldehyde acetonide obtained from L-ascorbic acid (Hubschwerlen, C. Synthesis 1986, 962; Takano, S. etal Heterocycles 1982, 19, 32) using the same procedure followed for the synthesis of 10 (Gung, B. W. etal J. Org. Chem. 2003, 68, 5956-5960). Reaction of 9 with the Li-acetylide prepared from 13 following the same method as described in Scheme 5 gave the adduct 14 with excellent diastereoselectivity. Conversion of 14 into the desired product 3 (R²=Me) followed the same steps as described in Scheme 5.

Synthesis of 4A (R =Me)

The eight steps involved in the synthesis of 4 A(R²=Me) from 14 (R²=Me) are shown in details in Scheme 8. The hydroxyl group of 14 (R²=Me) in Scheme 7 was oxidized to a ketone that was then reduced using K-selectride to get the hydroxyl-bearing center inverted using the same method described in Scheme 6. The resulting product 15 (R²=Me) was finally converted into the target molecule 4 A (R²=Me) following the same six steps that were used in Scheme 5 to transform 11 into 1. 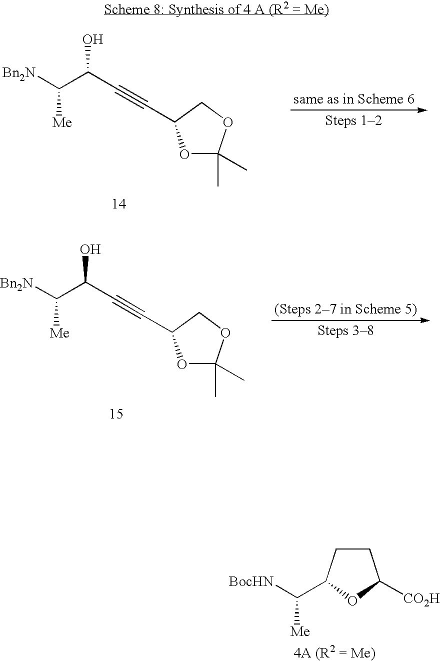
Synthesis of 5-8 (R=Me)

Compounds 5-8 (R²=Me) were synthesized starting with D-Ala derived N,N-dibenzylalaninal ((Reetz, M. T. etal Org. Synth. 1998, 76, 110; Reetz, M. T. Chem. Rev. 1999, 99, 1121-1162) following the same chemistry described for the synthesis of L-Ala based products 1- 4A (R²=Me) as shown in Schemes 5-8.

Synthesis of 18 (R²=Me; P,P=—C(Me)₂—)

The strategy adopted for the synthesis of the 3,4-dihydroxylated versions of the C₆-substituted furanoid sugar amino acids 18 as shown in Scheme 4 is elaborated in Scheme 9 with the details shown in each step of the protocol using a representative example with R²=Me and P,P=—C(CH₃)₂—. The intermediate 14 (R²=Me) from Scheme 7 was selectively reduced into a cis-allylic alcohol by hydrogenation using Lindlar's catalyst.

The resulting Z-olefinic compound 16 (R²=Me) was transformed into the cyclised intermediate 17 following the same methods described in Scheme 5.

Compound 17 was subjected to cis-hydroxylation using a catalytic amount of OsO₄ in the presence of N-methylmorpholine N-oxide (NMO) to introduce the hydroxyl groups at C3 and C4 positions with excellent diastereoselectivity leading to the formation of the triol 21. Next. The triol 21 was converted into the Boc-protected intermediate 22 following the same methods described in steps 2 and 3 in Scheme 5.

Routine functional group manipulations transformed 22 into compound 23 in three steps: selective protection of the primary hydroxyl, acetonide protection of the 3,4-dihydroxyl moiety and eventually silyl deprotection to free the primary hydroxyl group. The primary hydroxyl group of compound 23 was next oxidized to get the desired final product 18 (R²=Me, P,P=—C(Me)₂—) following a two-step oxidation protocol used in steps 6 and 7 in Scheme 5. 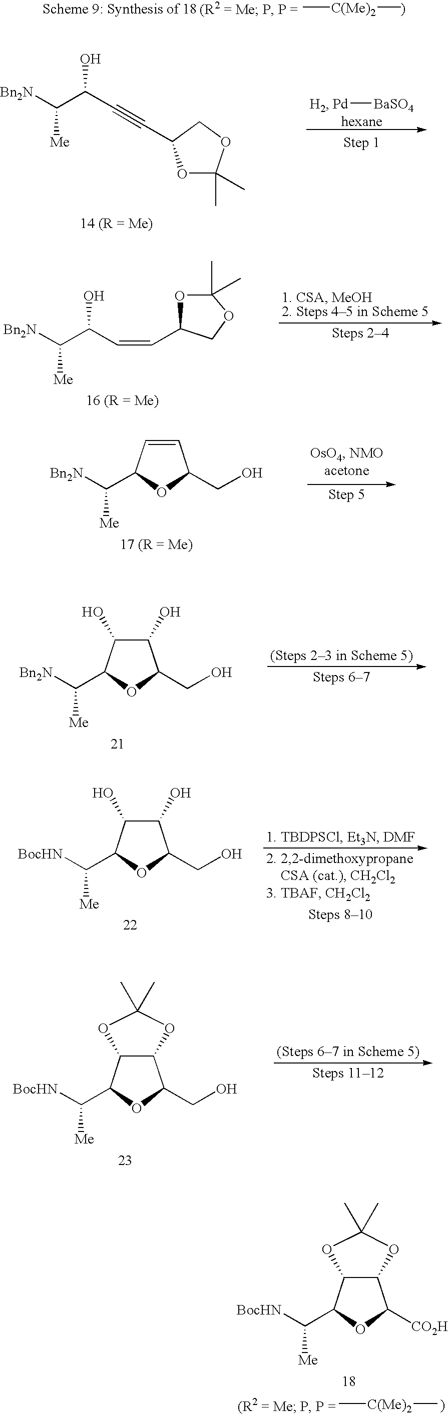
Step-1

A solution of compound 14 (7 g) in hexane (18 mL) was treated with Lindlar's catalyst (18 mg) and the resulting suspension was stirred under hydrogen atmosphere using a hydrogen-filled balloon for 30 minutes. The mixture was filtered through a short pad of Celite and the filtrate was concentrated under reduced pressure. The residue was purified by flash chromatography to afford compound 16 (6.2 g, 88% yield) as colourless oil.

Data for 16: R_{f}=0.45 (silica, 1:3 ethyl acetate/hexane); ¹H NMR (200 MHz, CDCl₃): δ 7.33-7.18 (m, 10H, ArH), 5.64 (dd, J=8.1, 11.7 Hz, 1H, C3H), 5.57 (dd, J=7.3, 11.7 Hz, 1H, C4H), 4.79 (m, 1H, C2H), 4.31 (m, 1H, C5H), 4.05 (dd, J=5.8, 8 Hz,1H, C1H), 3.79 (ABq, 2H, NCH₂Ph), 3.47 (m, 1H, C1H′), 3.42 (Abq, 2H, NCH₂Ph), 2.89 (dq, J=6.5, 7.3 Hz, 1H, C₆H), 1.39 and 1.32 (two s, 6H, acetonide methyls), 1.16 (d, J=6.5 Hz, 3H, C6CH₃).

Steps 2-4

To a solution of compound 16 (4 g) in MeOH (30 mL) at 0° C., camphorsulphonic acid (CSA) (4.87 g) was added portions-wise and stirred for 8 h at room temperature. The reaction was quenched with solid NaHCO₃ (1.76 g) and the MeOH was evaporated under reduced pressure. Then water was added to the residue and the mixture was extracted with EtOAc. The combined organic extracts were washed with brine, dried (Na₂SO₄) and concentrated in vacuo. Purification by column chromatography afforded the triol (3.4 g, 94%) as colourless oil.

The triol was transformed in two steps, following the same procedure as described in steps 4-5 in Scheme 5, into 17 (2 g, 94%) as colourless oil. Data for 17: R_{f}=0.45 (silica, 1:3 ethyl acetate/hexane); ¹H NMR (300 MHz, CDCl₃): δ 7.37-7.15 (m, 10H, ArH), 6.01 (dd, J=1.1, 5.2 Hz, 1H, C3H), 5.69 (dd, J=1.5, 5.2 Hz, 1H, C4H), 4.93-4.79 (m, 2H, C2 and C5), 3.81 (Abq, 2H, NCH₂Ph), 3.61 (dd, J=3, 11.3 Hz, 1H, C1H′) 3.52-3.43 (ABq, 2H, NCH₂Ph), 3.45 (dd, J=5.2, 11.3 Hz, 1H, C1H′), 2.79 (m, 1H, C6H), 1.1 (d, J=6.7 Hz, 3H, C6CH₃).

Step-5

To a solution of compound 17 (1.7 g) in acetone (10 mL) and H₂O (5 mL), N-methyl morpholine N-oxide (0.78 g) was added followed by catalytic amount of OSO₄. The reaction mixture was stirred for 36 h at room temperature. Acetone was evaporated and the mixture was extracted with EtOAc. The combined organic layers were washed with brine, dried (Na₂SO₄) and concentrated. Purification by column chromatography afforded compound 21 (1.8 g, 98% yield) as colourless oil. Data for 21: R_{f}=0.45 (silica, 4:1 ethyl acetate/hexane); ¹H NMR (200 MHz, CDCl₃): δ 7.37-7.2 (m, 10H, ArH), 3.94-3.7 (m, 5 H, NCH₂Ph, C2H, C3H, C4H), 3.64 (dd, J=2.9, 11.8 Hz, 1H, C1H), 3.48 (dd, J=4.4, 11.8 Hz, 1H, C1H′), 3.37 (ABq, 2H, NCH2Ph) 3.26 (m, 1H, C5H), 2.81 (m, 1H, C6H), 1.19 (d, J=6.6 Hz, 3H, C6CH₃).

Steps 6 and 7

A solution of 21 (1.4 g) in MeOH (8 mL) was hydrogenated on 20% Pd(OH)₂/C (100 mg) in acidic medium (1 drop of 6N HCl) with stirring at room temperature for 12 h. The Pd(OH)₂ was filtered off and washed with MeOH (20 mL). The filterate and washings were combined and concentrated under reduced pressure. The residue was dissolved in MeOH (10 mL) and cooled to 0° C. Then it was neutralised with Et₃N (1.1 mL) and di-tert-butyl dicarbonate (1 mL) was added and stirred for 3 h at room temperature. The reaction mixture was quenched with saturated NH₄Cl solution. The MeOH was removed under reduced pressure. The aqueous layer was extracted with EtOAc. The combined organic layers were washed with water, brine, dried (Na₂SO₄) and concentrated at vacuo. Purification by column chromatography afforded compound 22 (0.9 g, 82% yield) as a colourless oil. Data for 22: R_{f}=0.45 (silica, 1:9 MeOH/CHCl₃); ¹H NMR (200 MHz, CDCl₃): δ 4.67 (br, 1H, NH), 4.03 (m, 2H), 3.9-3.58 (m, 5H), 2-95 (br, 1H, OH), 2.73 (br, 1H, OH), 1.43 (s, 9H, t-butyl), 1.18 (d, J=6.5 Hz, 3H, C6CH₃).

Steps 8-10

To a solution of compound 22 (0.8 g) in DMF (5 ml) at 0° C., Et₃N (0.8 mL) was added followed by DMAP (35 mg). The reaction was stirred at room temperature for 6 h. The reaction was quenched with saturated NH₄Cl, and the mixture was extracted with EtOAc. The combined organic layers were washed with water, brine, dried (Na₂SO₄), and concentrated. Purification by column chromatography afforded TBDPS-protected compound (1.43 g 96% yield) as a colourless oil.

The resulting TBDPS-protected compound (1.2 g) was dissolved in CH₂Cl₂ (5 mL), cooled to 0° C. Then 2,2-dimethoxypropane (0.57 mL) was added followed by the addition of CSA (54 mg). The reaction mixture was stirred at room temperature for 8 h. The reaction was quenched with saturated NaHCO₃ solution and extracted with EtOAc. The combined organic layers were washed with water, brine, dried (Na₂SO₄) and concentrated. Purification by column chromatography afforded acetonide protected compound (1.25 g 97% yield).

The resulting acetonide protected compound (1.1 g) was dissolved in THF (5 mL) and cooled to 0° C. Then TBAF (2.3 mL, 1M in THF) was added and stirred for 3 h at room temperature. The reaction mixture was quenched with saturated NH₄Cl and the mixture was extracted with EtOAc. The combined organic layers were washed with brine, dried (Na₂SO₄) and concentrated in vacuo. Purification by column chromatography afforded compound 23 (0.62 g, 98% yield) as a colourless oil. Data for 23: R_{f}=0.45 (silica, 1:1.5 ethyl acetate/hexane); ¹H NMR (500 MHz, CDCl₃): δ 4.59 (dd, J=4.6, 7.0 Hz, 1H) 4.57 (m, 1H) 4.54 (dd, J=4.6, 6.4 Hz, 1H), 4 (dd, J=4.1, 7.6 Hz, 1H), 3.87-3.8 (m, 2H), 3.78 (dd, J=4.6, 5.8 Hz, 1H), 3.66 (dd J=4.1, 12.3 Hz, 1H), 1.53 (s, 3H, acetonide Me), 1.45 (s, 9H, t-butyl), 1.33 (s, 3H, acetonide Me), 1.19 (d, J=6.4 Hz, 3H, C6CH₃).

Steps 11 and 12

The primary hydroxyl group of compound 23 was next oxidized to get the desired final product 18 (R²=Me, P,P=—C(Me)₂—) following a two-step oxidation protocol used in steps 6 and 7 in Scheme 5.

The present invention is described in detail in the following examples which are provided by way of illustration only and therefore should not be construed to limit the scope of the invention.

We claim:

1) A chiral furanoid sugar amino acids and their salts of peptide compound carrying an additional chiral centre at C6 position with substituents, having a general structure as shown in FIG. 1, 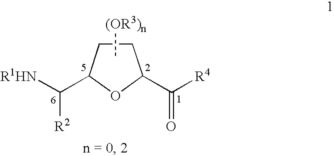
Wherein;
R¹=H, tert-butoxycarbonyl (Boc), benzyloxycarbonyl (Cbz), 9-fluroenylmethyl (Fmoc), acetyl or salts such as hydrochloric acid (HCl), tri fluro accetic acid (TFA),
R²=CH₃—, (CH₃)₂CH—, (CH)₂CHCH₂—, CH₃CH₂CH(CH₃)—, alkyl groups, (OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁵)CH₂—, (CO₂R⁵)CH₂CH₂—, Ph-, Ar-, PhCH₂—, ArCH₂—, Phenylalkyl-, arylalkyl-, (indolyl)CH₂—, (imidazolyl)CH₂—, and all other amino acid side-chains,
R³=H, tert-butyl, alkyl, benzyl, aryl-CH₂, CO(alkyl), CO(arylalkyl), SO₃H, PO₃H₂, silyl,
R⁴-O-alkyl, —O-arylalkyl, -amine, -alkylamine, -arylalkylamine,
R⁵=H, tert-butyl, alkyl, benzyl, aryl-CH₂, and others
R¹-R²=—(CH₂)ₙ— (n=2, 3, 4)

2) A chiral furanoid sugar amino acids as claimed in claim 1, wherein if the stereochemistry of C₆ is S form and the substitutions are R¹=Boc, R²=Me and R⁴=Me compound having structural formula 2

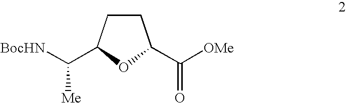

3) A chiral furanoid sugar amino acids as claimed in claim 1, wherein if the stereo chemistry of C₆ is S form and the substitutions are R¹=Boc, R²=Me and R⁴=H compound having structural formula 3

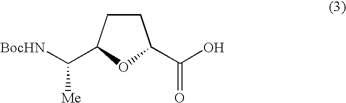

4) A chiral furanoid sugar amino acids as claimed in claim 1, wherein if the stereochemistry of C₆ is S form and the substitutions are and R¹=CF₃COOH.H, R²=Me and R⁴=Me compound having structural formula 4

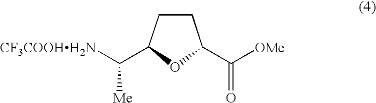

5) A chiral furanoid sugar amino acids as claimed in claim 1, wherein if the stereochemistry of C₆ is S form and the substitutions are and R¹=CF₃COOH.H, R²=Me and R¹=H compound having structural formula 5

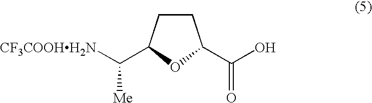

6) A chiral furanoid sugar amino acids as claimed in claim 1, wherein if the stereochemistry of C₆ is R form and the substitutions are and R¹=Boc, R²=Me and R⁴=Me compound having structural formula 6

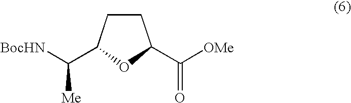

7) A chiral furanoid sugar amino acids as claimed in claim 1, wherein if the stereochemistry of C₆ is R form and the substitutions are R¹=Boc, R²=Me and R⁴=H compound having structural formula 7

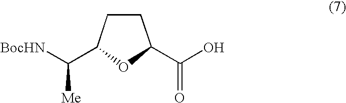

8) A chiral furanoid sugar amino acids as claimed in claim 1, wherein if the stereochemistry of C₆ is R form and the substitutions are R¹=CF₃COOH.H, R²=Me and R⁴=Me compound having structural formula 8

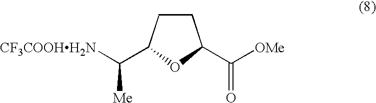

9) A chiral furanoid sugar amino acids as claimed in claim 1, wherein if the stereochemistry of C₆ is R form and the substitutions are R¹=CF₃COOH.H, R²=Me and R⁴=H compound having structural formula 9

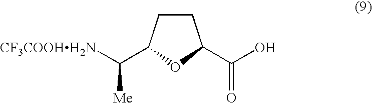

10) A chiral furanoid sugar amino acids as claimed in claim 1, wherein if the stereochemistry of C₆ is S form and the substitutions are R¹=Boc, R²=Me and R⁴=Me compound having structural formula 10

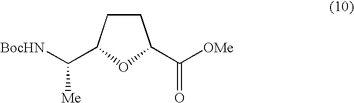

11) A chiral furanoid sugar amino acids as claimed in claim 1, wherein if the stereochemistry of C₆ is S form and the substitutions are R¹=Boc, R²=Me and and R⁴=H compound having structural formula 11

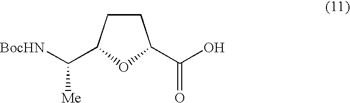

12) A chiral furanoid sugar amino acids as claimed in claim 1, wherein if the stereochemistry of C₆ is S form and the substitutions are R¹=CF₃COOH.H, R²=Me and R⁴=Me compound having structural formula 12

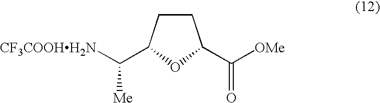

13) A chiral furanoid sugar amino acids as claimed in claim 1, wherein if the stereochemistry of C₆ is S form and the substitutions are R¹=CF₃COOH.H, R²=Me and R⁴=H compound having structural formula 13

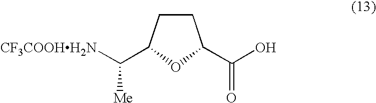

14) A chiral furanoid sugar amino acids as claimed in claim 1, wherein if the stereochemistry of C₆ is R form and the substitutions are R¹=Boc, R²=Me and R⁴=Me compound having structural formula 14

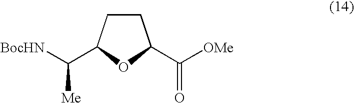

15) A chiral furanoid sugar amino acids as claimed in claim 1, wherein if the stereochemistry of C₆ is S form and the substitutions are R¹=Boc, R²=Me and R⁴=H compound having structural formula 15

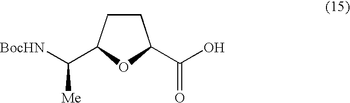

16) A chiral furanoid sugar amino acids as claimed in claim 1, wherein if the stereochemistry of C₆ is R form and the substitutions are R¹=CF₃COOH.H, R²=Me and R⁴=Me compound having structural formula 16

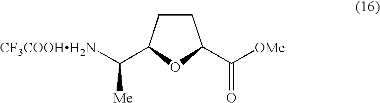

17) A chiral furanoid sugar amino acids as claimed in claim 1, wherein if the stereochemistry of C₆ is R form and the substitutions are R¹=CF₃COOH.H, R²=Me and R⁴=H compound having structural formula 17

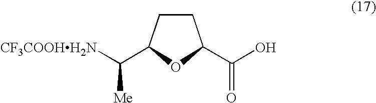

18) A chiral furanoid sugar amino acids as claimed in claim 1, wherein if the stereochemistry of C₆ is S form and the substitutions are R¹=Boc, R²=Me and R⁴=Me compound having structural formula 18

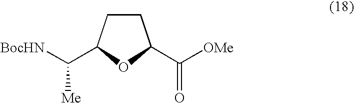

19) A chiral furanoid sugar amino acids as claimed in claim 1, wherein if the stereochemistry of C₆ is S form and the substitutions are R¹=Boc, R²=Me and R⁴=H compound having structural formula 19

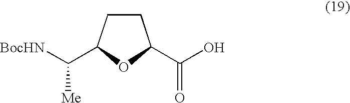

20) A chiral furanoid sugar amino acids as claimed in claim 1, wherein if the stereochemistry of C₆ is R form and the substitutions are R¹=CF3COOH.H, R²=Me and R⁴=Me compound having structural formula 20

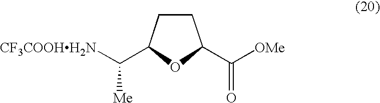

21) A chiral furanoid sugar amino acids as claimed in claim l, wherein if the stereochemistry of C₆ is S form and the substitutions are R¹=CF₃COOH.H, R²=Me and R⁴=H compound having structural formula 21

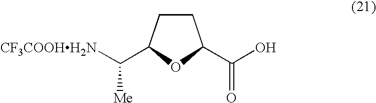

22) A chiral furanoid sugar amino acids as claimed in claim 1, wherein if the stereochemistry of C₆ is R form and the substitutions are R¹=Boc, R²=Me and R⁴=Me compound having structural formula 22

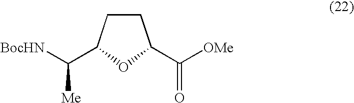

23) A chiral furanoid sugar amino acids as claimed in claim l, wherein if the stereochemistry of C₆ is R form and the substitutions are R¹=Boc, R²=Me and R⁴=H compound having structural formula 23

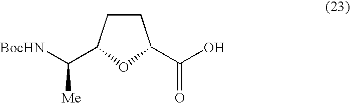

24) A chiral furanoid sugar amino acids as claimed in claim 1, wherein if the stereochemistry of C₆ is R form and the substitutions are R¹=CF₃COOH.H, R²=Me and R⁴=Me compound having structural formula 24

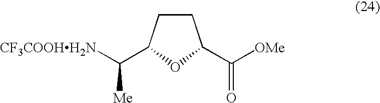

25) A chiral furanoid sugar amino acids as claimed in claim 1, wherein if the stereochemistry of C₆ is R form and the substitutions are R¹=CF₃COOH.H, R²=Me and R⁴=H compound having structural formula 25

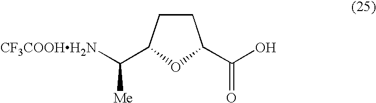

26) A chiral furanoid sugar amino acids as claimed in claim 1, wherein if the stereochemistry of C₆ is S form and the substitutions are R¹=Boc, R²=Me and R⁴=Me compound having structural formula 26

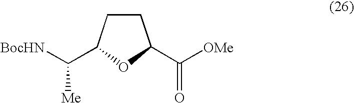

27) A chiral furanoid sugar amino acids as claimed in claim 1, wherein if the stereochemistry of C₆ is S form and the substitutions are R¹=Boc, R²=Me and R⁴=H compound having structural formula 27

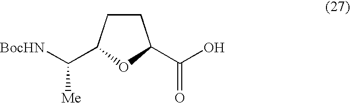

28) A chiral furanoid sugar amino acids as claimed in claim 1, wherein if the stereochemistry of C₆ is S form and the substitutions are R¹=CF₃COOH.H, R²=Me and R⁴=Me compound having structural formula 28

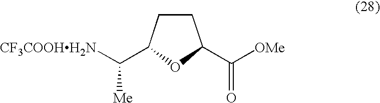

29) A chiral furanoid sugar amino acids as claimed in claim 1, wherein if the stereochemistry of C₆ is S form and the substitutions are R¹=CF₃COOH.H, R²=Me and R⁴=H compound having structural formula 29

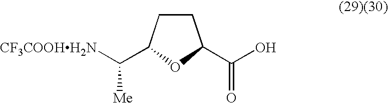

30) A chiral furanoid sugar amino acids as claimed in claim 1, wherein if the stereochemistry of C₆ is R form and the substitutions are R¹=Boc, R²=Me and R⁴=H compound having structural formula 30

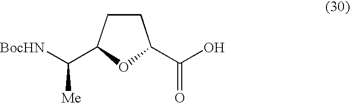

31) A chiral furanoid sugar amino acids as claimed in claim 1, wherein if the stereochemistry of C₆ is R form and the substitutions are R¹=CF₃COOH.H, R²=Me and R⁴=Me compound having structural formula 31

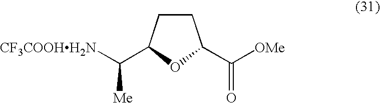

32) A chiral furanoid sugar amino acids as claimed in claim 1, wherein if the stereochemistry of C₆ is R form and the substitutions are R¹=CF₃COOH.H, R²=Me and R⁴=H compound having structural formula 32

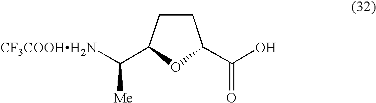

33) A chiral furanoid sugar amino acids as claimed in claim 1, wherein if the stereochemistry of C₆ is S form and the substitutions are R¹=Boc, R²=Me, R³=CO(alkoxy) and R⁴=Me compound having structural formula 33

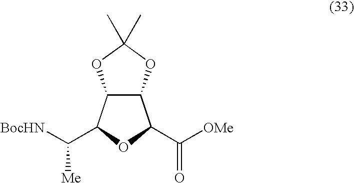

34) A chiral furanoid sugar amino acids as claimed in claim 1, wherein if the stereochemistry of C₆ is S form and the substitutions are R¹=Boc, R²=Me, R³=R³=CO(alkoxy) and R⁴=H compound having structural formula 34

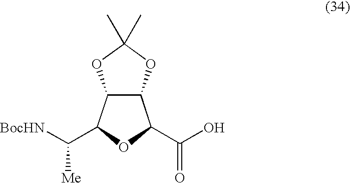

35) A chiral furanoid sugar amino acids as claimed in claim 1, wherein if the stereochemistry of C₆ is S form and the substitutions are R¹=CF₃COOH.H, R²=Me R³=H and R⁴=Me compound having structural formula 35

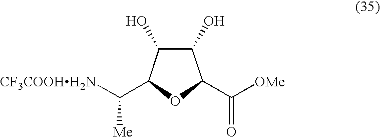

36) A chiral furanoid sugar amino acids as claimed in claim 1, wherein if the stereochemistry of C₆ is S form and the substitutions are R¹=CF₃COOH.H, R²=Me R³=H and R⁴=H compound having structural formula 36

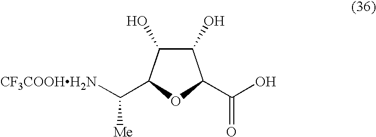

37) A chiral furanoid sugar amino acids as claimed in claim 3, wherein compound having following characteristics R_{f}=0.45 (silica, 1:3 ethyl acetate/hexane): ¹H NMR (200 MHz, CDCl₃): δ 4.67 (d, J=6.6, 1H, NH), 4.54 (dd, J=5.2, 8.1 Hz, 1H, C2H), 4.15 (m, 1H, C5H), 3.74 (s, 3H, CO₂Me), 3.71 (m, 1H, C6H), 2.28 (m, 1H), 2.03 (m 1H), 1.72 (m, 2H) 1.44 (s, 9H, t-butyl) 1.14 (d, J=6.6 Hz, 3H, C6CH₃).

38) A process for preparing C6-substituted chiral furanoid sugar amino acids of peptide compound, carrying natural amino acid side-chains and having a general structure 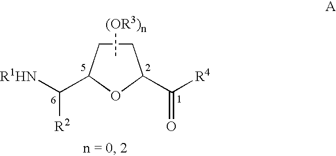
Wherein;
R¹=H, tert-butoxycarbonyl (Boc), benzyloxycarbonyl (Cbz), 9-fluroenylmethyl (Fmoc), acetyl or salts such as hydrochloric acid (HCl), tri fluoro acetic acid (TFA),
R²=CH₃—, (CH₃)₂CH—, (CH₃)₂CHCH₂—, CH₃CH₂CH(CH₃)—, alkyl groups, (OR³)CH₂—, CH₃(OR³)CH—, (R³S)CH₂—, CH₃SCH₂CH₂—, (RHN)CH₂CH₂CH₂CH₂—, (CONH₂)CH₂—, (CONH₂)CH₂CH₂—, (CO₂R⁵)CH₂—, (CO₂R⁵)CH₂CH₂—, Ph-, Ar-, PhCH₂—, ArCH₂—, Phenylalkyl-, arylalkyl-, (indolyl)CH₂—, (imidazolyl)CH₂—, and all other amino acid side-chains,
R³=H, tert-butyl, alkyl, benzyl, arylCH₂, CO(alkyl), CO(arylalkyl), SO₃H, PO₃H₂, silyl,
R⁴=—O-alkyl, —O-arylalkyl, -amine, -alkylamine, -arylalkylamine, and others
R⁵=H, tert-butyl, alkyl, benzyl, arylCH₂,
R¹-R²=—(CH₂)ₙ— (n=2,3,4)
said process comprising the steps of:
a) addition of L- or D-N,N-dibenzylamino aldehydes, prepared in-situ by reacting 3,4-O-isopropylidene-1,1-dibromobut-1-en-3,4-diol with n-BuLi, to the N,N-dibenzyl amino aldehyde to give the propargylic alcohol adducts, 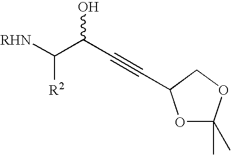
b) hydrogeneation of the adduct obtained in step (a) in presence of Pd(OH)₂—C catalyst and in the presence of acid to deprotect (i) N-terminus, (ii) acetonide and (iii) reduce the triple bond, all in one pot and to get an intermediate of N-Boc-protected,
(c) intermediate of step (b) dissolved in MeOH, neutralize with Et₃N and Bo.C₂O and stirring the same for a period of 3 hours to obtain triol as a intermediate compound, 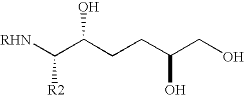
(d) mixing 2,4,6 tri iso propyl benzene chloride with triol of step (c) to obtain sulfonated intermediate,
(e) mixing sulfonated intermediate of step (d) with water in presence of MeOH and K₂CO₃, extract the same in EtOAc, 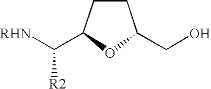
(f) dissolving intermediate compound of step (e) in presence of CH₂Cl & DMSO and Et₃N/SO₃-Py complex, obtaining organic layer & aqueous layer, washing with water to obtain a brine solution.
(g) brine solution of step (f) dissolved in 2-methyl 2-Butene and t-BuOH & in presence of NaClO₂, NaHPO₄, acidifying with HCl to obtain desired product,

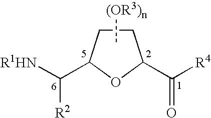

39) A process as claimed in claim 38 wherein in step (g), if C₆ is S form and R¹=Boc, R²=Me and R⁴=H compound having structural formula

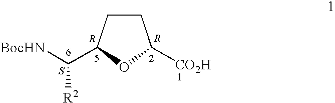

40) A process as claimed in claim 38, wherein if C₆ is S form and R¹=Boc, R²=Me and R⁴=H compound 3 having structural formula

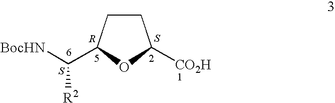

41) A process as claimed in claim 38, wherein if C₆ is R form and R¹=Boc, R²=Me and R⁴=H compound 5 having structural formula

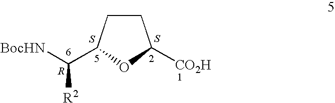

42) A process as claimed in claim 38, wherein if C₆ is S form and R¹=Boc, R²=Me and R⁴=H compound 7 having structural formula

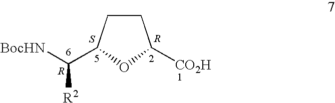

43) A process as claimed in claim 38 wherein said process comprising the steps of wherein if C₆ is S form and R¹=Boc, R²=Me and R⁴=H compound having structural formula

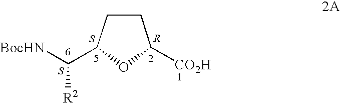

44) A process as claimed in claim 38, wherein intermediate compound obtained in step (a) is oxidized to a keto intermediate using SO₃-Py and reducing in presence of k-selectride to get the hydroxyl bearing centre inverted intermediate having structure (12), and following the steps (b) to (g) to obtain a compound 2, 4, 6 and 8.

45) A process as claimed in claimed in claim 44, wherein the intermediate structure 12 having general formula

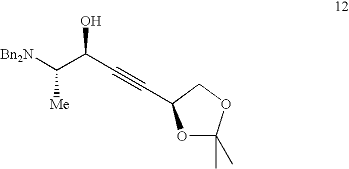

46) A process as claimed in claim 44, wherein if C₆ is S form and R¹=Boc, R²=Me and R⁴=H compound 2 having structural formula

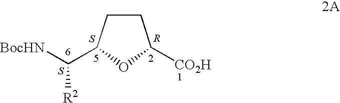

47) A process as claimed in claim 38, wherein if C₆ is S form and R¹=Boc, R²=Me and R⁴=H compound 4 having structural formula

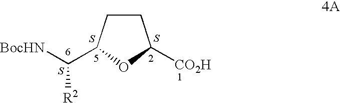

48) A process as claimed in claim 38, wherein if C₆ is S form and R¹=Boc, R²=Me and R⁴=H compound 6 having structural formula

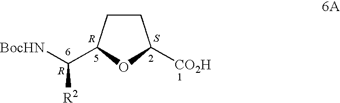

49) A process as claimed in claim 1, wherein if C₆ is R form and R¹=Boc, R²=Me and R⁴=H compound 8 having structural formula

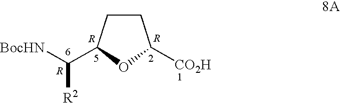

50) A Chiral furamn acid compound as claimed in claim 5, 9, 13, 17, 21, 25, 29, 33 and 37, wherein treatment with FmocOSu in dioxane-water (1:1) is carried out to give N-Fmoc protected C₆-substituted furanoid sugar amino acid.
